(19) United States
(12) Patent Application Publication
(10) Pub. No.: US 2021/0079399 A1
(43) Pub. Date: Mar. 18, 2021

(54) METHODS FOR THE TREATMENT OF PROSTATE CANCER

(71) Applicant: Cleveland State University, Cleveland, OH (US)

(72) Inventors: Girish C. Shukla, Cleveland, OH (US); Savita Singh, Cleveland, OH (US)

(21) Appl. No.: 16/938,242

(22) Filed: Jul. 24, 2020

Related U.S. Application Data
(60) Provisional application No. 62/877,910, filed on Jul. 24, 2019.

(51) Int. Cl. C12N 15/113; A61P 35/00; A61K 31/4166; A61K 31/7088

(52) U.S. Cl. CPC C12N 15/1135; A61P 35/00; C12N 2320/31; A61K 31/7088; C12N 2310/141; A61K 31/4166

(57) ABSTRACT

The general inventive concepts contemplate methods and compositions for detecting, reducing, or treating therapeutic resistance associated with prostate cancer in an individual in need thereof. In certain exemplary embodiments, the method comprises administration of miR-149-5p.

### CROSS-REFERENCE TO RELATED APPLICATIONS

This application claims priority to and any benefit of U.S. Provisional Application 62/877,910, filed Jul. 24, 2019, the content of which is incorporated herein as if recited in its entirety.

### FIELD

The general inventive concepts relate to the field of medical therapies and more particularly to methods for the treatment or prevention of prostate cancer.

### BACKGROUND

Prostate cancer (PCa) is the most common cause of cancer in men, with 137.9 new cases per 100,000 men per year. The overall 5-year survival rate for prostate cancer is very high. However, up to 20% of men who undergo androgen deprivation treatment for prostate cancer develop castration-resistant prostate cancer (CRPC) within 5 years, with a very poor median survival of approximately 15 to 36 months for those with metastatic CRPC. With the advent of several new generation of therapeutics to treat CRPC, the challenge to overcome the relapse rate and increase survival after the treatment still remains a major issue. Androgen Deprivation Therapy (ADT) is often used to treat advanced PCa, however, despite high initial success rates of this therapy, recurrence to CRPC form is often unavoidable. One of the primary reasons responsible for this recurrence is persistently high intratumoral androgens and active Androgen Receptor(s) (AR), this occurs despite the low levels of circulating androgens resulting from ADT.

### SUMMARY

A need exists for treatment options for individuals having prostate cancer, including those who experience castration-resistant prostate cancer after androgen deprivation therapy.

In certain embodiments, the general inventive concepts are directed to a method of reducing or treating therapeutic resistance associated with prostate cancer in an individual in need thereof. In certain exemplary embodiments, the method comprises administration of miR-149-5p.

In an exemplary embodiment, the general inventive concepts contemplate a composition comprising miR-149-5p.

In an exemplary embodiment, the general inventive concepts contemplate a vector for introducing miR-149-5p for the treatment or prevention of prostate cancer.

Numerous other aspects, advantages, and/or features of the general inventive concepts will become more readily apparent from the following detailed description of exemplary embodiments and from the accompanying drawings being submitted herewith.

### BRIEF DESCRIPTION OF THE DRAWINGS

The general inventive concepts, as well as embodiments and advantages thereof, are described below in greater detail, by way of example, with reference to the drawings in which:

FIG. 1 shows the functional annotation of predicted targets of miR-149-5p by consensus pathway database: (A) Table indicating the major predicted miR-149-5p target genes contributing to key deregulated pathways in PCa. (B) Functional gene set overlap graph of the predicted target genes indicating an overlap between genes regulating cellular processes. The node size reflects the size of the gene set and the node color density is p-value.

FIG. 2 shows miR-149-5p expression analysis: (A) Normal prostate epithelium and cancer cell lines; (B) TCGA dataset meta-analysis; and (C), comparison of normal and patient tumor samples: miR-149-p expression was significantly downregulated in metastatic prostate cancer samples as compared to normal samples obtained from Prostate Cancer Tumor Network funded by the Department of Defense (n=10 for normal, n=10 each G3+3, G4+3 and CRPC samples). (n≥3; t-test ***P<0.001). Error bars, standard error of the mean (s.e.m).

FIG. 3 shows mRNA and protein levels of SREBF1 and FASN in LNCaP (AR dependent) and PC-3 (AR null) cells. Western blot analysis showing suppression of SREBP1 and FASN protein (A). Overexpression of miR-149-5p leads to the reduction of SREBF1 and FASN mRNA in miR-149-5p treated LNCaP and PC3 cells cell lines (B)

FIG. 4 shows mRNA and protein levels of HMGCS1, SCARB1 and HMGCR. In miR-149-5p treated LNCaP cells, downregulation of HMGCS1, HMGCR and SCARB1 expression was observed at mRNA levels (A) and protein levels (B).

FIG. 5 shows miR 149-5p inhibits the growth of PCa cells. Cell proliferation assay using BrDU incorporation in LNCaP cells transfected with 20 nM miR-149-5p mimic alone and in combination with enzalutamide.

FIG. 6 shows miR 149-5p inhibits the growth of PCa cells. Cell viability assay on LNCaP cells transfected with either miR 149-5p mimic (20 nM), and NC mimic (20 nM). The assay determines the amount of ATP in viable cells, which is quantitated using a luciferase reaction and measurement of luminescence signal (A). Western blot analysis for cleaved PARP in miR-149-5p and NC treated cells indicated induction of apoptosis in miR-149-5p treated LNCaP (8D) and PC-3 (8E) cells.

FIG. 7 shows miR-149-5p impedes proliferation and migration of cancer cells: the colony formation assay (Panel A) shows miR-149-5p transfected cells forming fewer colonies compared to NC transfected cells. The wound healing assay (Panel B) shows that the migration of cancer cells is markedly reduced in miR-149-5p transfected LNCaP cells as compared to negative control.

FIG. 8 shows miR-149-5p inhibits cancer cell invasion: The Matrigel invasion assay (Panel A—Invaded cells) shows that transient expression of miR-149-5p in LNCaP and 22rv1 cells inhibited the invasion. The number of cells in three random fields (40X) were counted for each group and plotted (B).

FIG. 9 shows (A) Immunoblot panels show the repression of endogenous AR protein expression and (B) mRNA, as determined by RT-qPCR in miR-149-5p transfected LNCaP, LAPC4 and 22RV1 cells. (n≥3; t-test ***P<0.001).

FIG. 10 shows that AR and SREBP1 are both a direct target of miR-149-5p: (A) The WT and see mutant. mRNA::miRNA duplex are shown in Red and Black letters. (B) Co-expression of miR-149-5p downregulated the expression of FFluc construct with WT AR 3′UTR (Bar-1, Panel B) and rescues the expression with mutant 3′UTR (Bar-2 Panel B), (C) The WT and see mutant. mRNA::miRNA duplex are shown in Red and Black letters. (D) Co-expression of miR-149-5p downregulated the expression of FFluc construct with WT AR 3′UTR (Bar-1, Panel B) and rescues the expression with mutant 3′UTR (Bar-2 Pnel B). (n≥3; t-test **P<0.01).

FIG. 11 shows miR-149-5p targets the AR transactivation and downregulates PSA expression both at mRNA and protein levels in LNCaP, LAPC4 and 22RV1 cells: miR-149-5p mediated targeting of AR negatively regulates AR transactivation capacity as demonstrated by downregulation of PSA expression, in both, protein LAPC4 (A), LNCaP (B), 22RV1 (C) and mRNA (supplemental). (n>3; t-test ***P<0.001, ** P<0.01, * P<0.01). Error bars, s.e.m.

FIG. 12 shows miR-149-5p suppresses 22RV1 xenograft tumor growth and potentiates Enzalutamide therapy: Xenograft mice representing PCa tumor growth in miR-149-4p treated, Enzalutamide treated and miR-1495p+enzalutamide treated animals, the tumor regresses significantly in miR-149-5p treated and miR-149-5p and enzalutamide treated animals as shown by the quantitative plot and tumor images taken 17 days after the beginning of treatment (8A).

FIG. 13 shows miR-149-5p suppresses AR, HMGCS1 and SCARB1 expression in xenograted tumors:qRT-PCR analysis of expression of miR-149-5p expression in tumors derived from the xenograft mice given various treatments (A). Western blot analysis for the expression of AR, HMGCs1 and SCARB1 in tumors derived from xenograft animals shows suppression of AR, HMGCs1 and SCARB1 in themiR-149-5p treated animals (B).

FIG. 14 shows miR-149-5p suppresses total intracellular cholesterol and testosterone levels in 22RV1 xenografts: analysis of total cholesterol in 22RV1 tumor xenograft grown in culture for 2 days(A) using red cholesterol amplex assay. Quantitative ELISA analysis total intracellular testosterone in 22 RV1 xenograft tumor shows reduced testosterone levels in miR-149-5p treated tumors (B).

FIG. 15 shows a model for miR-149-5p-AR and SREBP! Regulation pathways in regulating prostate cancer growth. Altered expression of SREBP1 promotes intratumoral steroidogenesis: SREBP1 is involved in the transcriptional regulation of AR and formation of fatty acid through altered expression of FASN. Both of which promote CRPC growth and feed in cholesterogenesis and lipogenesis. The schematic represents the constitutively active AR signaling and upregulated expression of SREBP1 leads to intratumoral androgen synthesis, controlling the expression of these proteins by miR-149-5p may significantly reduce intratumoral DHT by regulating influx and synthesis of cholesterol and also inhibit AR signaling by downregulating AR protein.

### DETAILED DESCRIPTION

While the general inventive concepts are susceptible of embodiment in many different forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

The materials, systems, and methods described herein are intended to be used to provide compositions and methods for the treatment of prostate cancer.

Prostate cancer growth and proliferation depends on androgen signaling mediated by transactivation of Androgen Receptor (AR). Androgen ablation remains the mainstay therapy for treatment of the disease. However, despite androgen ablation, the disease often relapses to a more aggressive form known as Castration-Resistant Prostate Cancer (CRPC). Androgen Signaling Inhibitor (ASI) such as Abiraterone Acetate and Enzalutamide are the most effective treatment methods for CRPC. However, more than one-third of CRPC patients eventually develop resistance to these treatments mostly due to the gain of function in the AR protein and increase in intratumoral dihydrotestosterone (DHT) synthesis. Intratumoral DHT synthesis from steroid precursors in tumors is augmented by up-regulation of intracellular cholesterol promoted by Sterol Regulatory Element-Binding Protein-1 (SREBP-1), HMGCS and HMGCR and influx of cholesterol promoted by SCARB1. Tumor specific downregulation of microRNAs which regulate the AR and steroid biosynthesis appears to promote tumor growth and resistance to therapeutics in CRPC.

Recently, it was discovered that the selective growth of tumor cells proficient in facilitating de novo androgen synthesis could lead to sustainable endogenous androgen concentrations that are sufficient for the tumors to proliferate. A common upstream precursor in the steroidogenic pathway is cholesterol, which has been studied in cancer biology predominantly for its role in maintaining membrane integrity and cell proliferation. De novo steroidogenesis by tumor cells has provided a new avenue for cholesterol research as modifications in cholesterol homeostasis could lead to PCa progression to CRPC during ADT.

SREBP-1 is a transcription factor that plays major role In PCa cholesterol homeostasis, SREBP1 is involved in the transcriptional regulation of androgen receptor (AR), formation of fatty acid(s) through altered expression of fatty acid synthase (FASN), the enzyme responsible for de novo synthesis of fatty acids and regulation of HMG CoA synthase (HMGCS), which is required for de novo cholesterol synthesis. Genetic overexpression or knockdown of SREBP-1 in prostate cancer cells results in corresponding increase or decrease in AR and FASN, demonstrating the significance of SREBP1 in regulating PCa growth. SREBP-1 has been shown to promote the growth, migration, invasion, and castration resistant progression of PCa cells in vitro and in vivo. Moreover, overexpression of SREBP1 induces overexpression of FASN. Tumors with increased FASN levels promote aggressive growth when compared with tumors with normal FASN expression. Therefore, SREBP1 promotes PCa growth via its role in AR signaling as well as independently. PCa tumors have been shown to accumulate higher levels of cholesterol when compared to the healthy tissues, potentially for its role in the intratumoral androgen synthesis. This intratumoral accumulation of cholesterol appears to be a consequence of both de novo synthesis, regulated by HMGCS and lipoprotein-mediated uptake by scavenger receptor class B member I (SCARB1), high expression of SCARBI in metastatic prostate cancer almost always acquires resistance to androgen depletion.

The general inventive concepts are based, in part, on the finding that increased levels of SCARBI are required for transport of cholesterol into the tumor cell. This uptake of cholesterol can be exploited by the tumor cells as another means of facilitation androgen synthesis. This is potentially evident from reported decreased plasma HDL cholesterol levels found in several groups of cancer patients representing a broad range of malignancies. A better understanding of the role of cholesterol homeostasis and function of SREBP1 as key regulator of cholesterol homeostasis with its interconnected role in AR signaling has led to considerable investigation in exploring new avenues for adjuvant therapies.

MicroRNAs have been studied to control cellular processes in a spatiotemporal manner, large body of recent research indicated the function of microRNAs is to intricately regulate cellular processes by targeting multiple key proteins in a network. microRNAs can be used as a therapy or as an aid to an appropriate current AR targeting therapy for enhancing the positive effects of the treatment. Fine tuning the cholesterogenesis and AR signaling with microRNAs is a reasonable strategy.

The general inventive concepts are based, in part, on the understanding that microRNAs are capable of fine tuning the gene expression of multiple key proteins involved in cancer progression, therefore tumors often display deregulation in microRNA expression as a mean to constitutively activate survival pathways. In this disclosure, Applicants demonstrate the tumor suppressive function of miR-149-5p in PCa growth. miR-149-5p is coded in intron one of Glypican 1 (GPC-1) gene (24). miR-149-5 has been shown to target GIT1 in breast cancer and regulate metastasis, and it has been documented to be downregulated in tumors from patients with early recurrence. Downregulation of miR-149-5p has been recently shown to promote Acute Myeloid Leukemia and it was shown to target Fas ligand and induce apoptosis in the same model. Computational analysis show that miR-149-5p is expected to target genes in AR signaling, metabolic signaling by SREBP1 and steroid biosynthesis. Several other microRNAs have been investigated in PCa however no recent report has indicated one single microRNA capable of regulating AR and SREBP1 signaling together, which is critical in AR-V7 driven tumors as well as in AR independent CRPC growth. Applicants further demonstrate that restoration of miR-149-5p expression can abolish the intratumoral androgen synthesis by regulating cholesterogenesis and R signaling. Thus, the general inventive concepts contemplate compositions and methods comprising miR-149-5p for reducing or modulating intratumoral androgen synthesis by regulating cholesterogenesis. The compositions and methods may aid in enhancing the efficacy of current therapeutics in PCa.

have investigated the role of miR-149-5p for its anti-proliferative potential in PCa. Applicants have observed down-regulation of miR-149-5p in prostate tumors and discovered that it targets key factors in genomic and non-genomic AR signaling pathways. Ectopic expression of miR-149-5p inhibits invasion and proliferation, alone as well as in combination with AR antagonist Enzalutamide. Applicants further explored the regulation of AR signaling and cholesterogenesis by miR-149-5p. Our results indicate a significant suppression of intracellular cholesterol and testosterone in tumors treated with miR-149-5p indicating its tumor suppressive function. The present disclosure demonstrates that miR-149-5p may serve as an adjuvant therapeutic agent for CRPC in combination with ASIs.

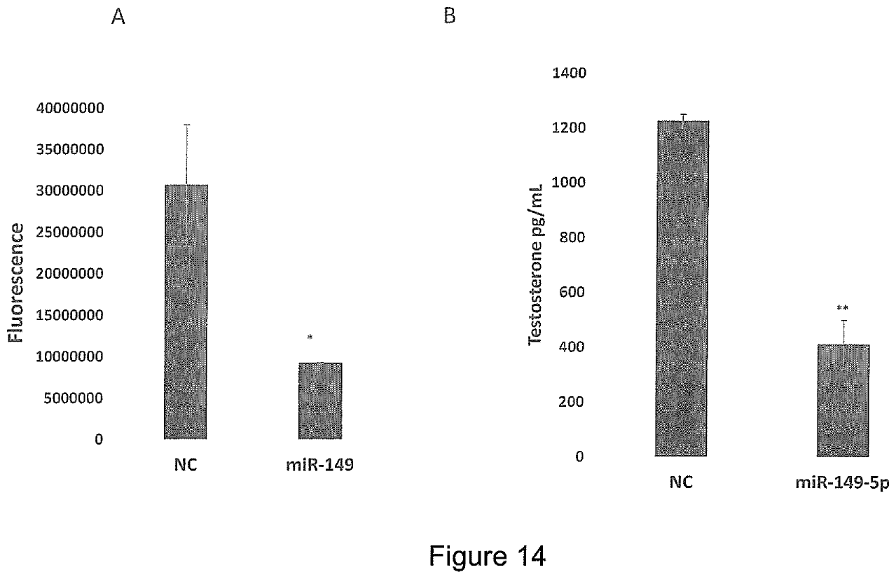

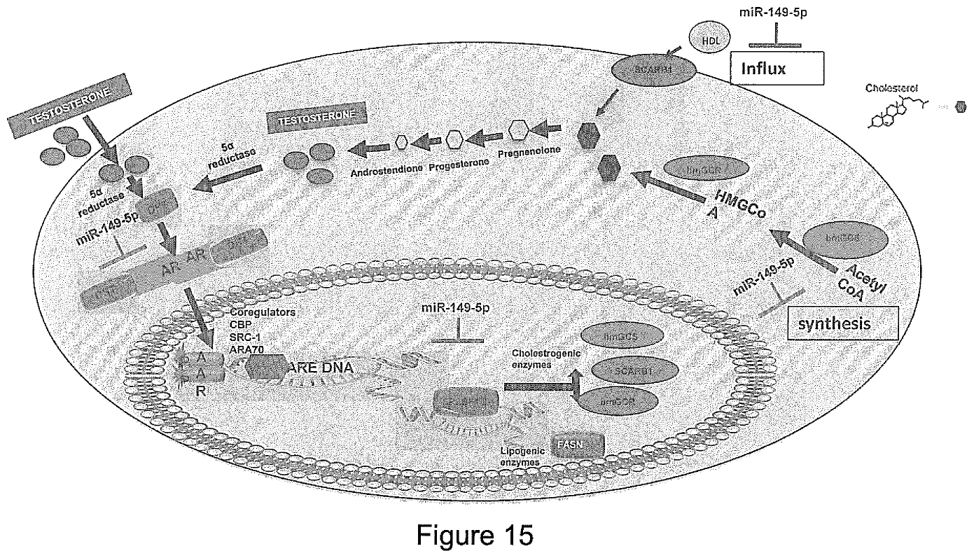

As previously discussed, the general inventive concepts are based, in part, on the understanding that restoration of androgen receptor signaling and induction of intratumoral androgen synthesis with SREBP1 as one of the key transcriptional factor promoting AR activation and androgen synthesis accounts for relapse of PCa. Additionally, aberrant cholesterol regulators, receptors, and transporters contribute to cholesterol accumulation, lethal and higher-grade Gleason score PCa has been shown to rely on de novo cholesterol synthesis. Numerous molecular alterations are responsible for this change, including the dysregulation of microRNAs. Applicants have discovered that miR-149-5p is a tumor suppressive microRNA in PCa. Our results indicate miR-149-5p targets AR and SREBP1, two major transcript factors. Applicants discovered that miR-149-5p expression is suppressed in PCa, which may contribute to sustained androgen receptor signaling. miR-149-5p significantly reduced SREBP1, HMGCR, HMGCs and SCARB1 expression in vitro as well as in vivo model and decreased PCa tumor growth in xenograft mice model. miR-149 inhibits PCa cell proliferation and shows synergistic effect with enzalutamide and miR-149-5p decreases the levels of intracellular cholesterol in xenograft tumors. AS demonstrated in the model (FIG. 14) regulation of SREBP1 and AR by miR-149-5p may suppress PCa growth by regulating AR signaling and intracellular pool of available cholesterol which acts as a precursor for DHT synthesis.

Accordingly, the general inventive concepts relate to new understanding of PCa progress and resistance. The general inventive concepts provide a powerful tool for modulation of PCa progression and, more particularly, the general inventive concepts are directed to compositions and methods for reduction or interruption of certain biological processes associated with prostate cancer, such methods and compositions comprise miR-149-5p, and its use.

In an exemplary embodiment, the general inventive concepts contemplate a method for providing gene therapy to an individual in need of prostate cancer therapy.

In certain exemplary embodiments, the general inventive concepts are directed to an expression vector comprising a first nucleic acid molecule. In an exemplary embodiment, the general inventive concepts contemplate a vector for introducing at least one microRNA molecule.

In certain exemplary embodiments, the expression vector comprises miR-149-5p or a nucleic acid molecule encoding for miR-149-5p.

### EXAMPLES

The following examples describe various compositions and methods for the modulation of various biological processes involved in prostate cancer progression and associated therapeutic resistance.

miRNA microarray profiling identified miR-149-5p differentially expressed in the in vivo-selected metastatic prostate cancer cells versus normal prostate epithelial cells. The microRNAs in LNCaP C4-2B and PrEC and PrSC cells were deep sequenced to investigate deregulation of microRNAs and identified miR-149-5p as one of the downregulated microRNAs.

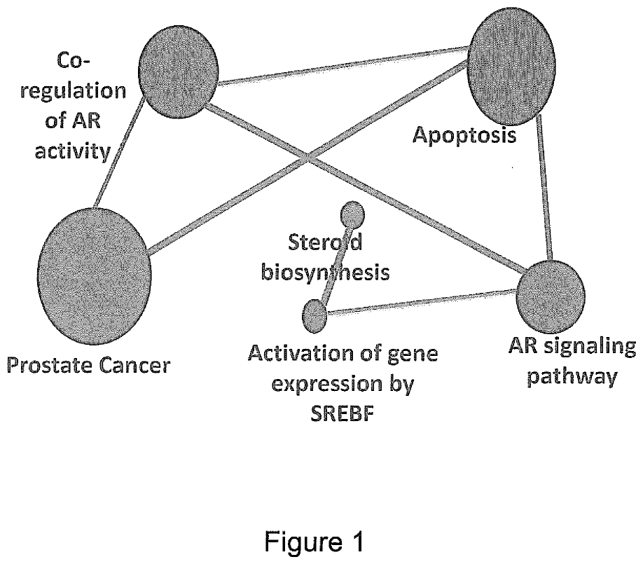

Functional annotation of predicted targets of miR-149-5p by consensus pathway database: In order to investigate the significance of miR-149-5p downregulation in PCa progression Applicants pooled out the predicted target genes of miR-149-5p from miRecord with selection criteria of a minimum of 4 prediction tools predicting the same targets. These genes were analyzed for their role in pathways critical for PCa growth. Pathways involved in PCa including AR and SREBF1 signaling pathways as well as steroid biosynthesis pathways were highly significant pathways predicted to be regulated by miR-149-5p (FIG. 1). Applicants further investigated if miR-149-5p alone has the potential to regulate these predicted pathways which largely govern AR dependent as well as AR independent PCa growth.

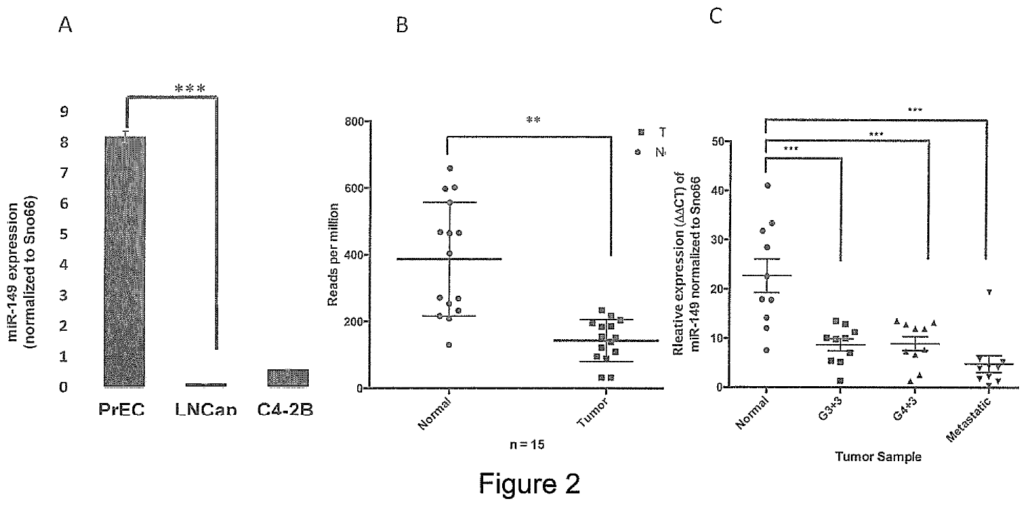

Downregulation of miR-149-5p in cell lines, TCGA dataset and PCa tumor samples: Applicants extracted data from openly available TCGA database using ucsc xena and discovered that miR-149-5p is significantly downregulated in tumor samples (FIG. 2B) miR-149-5p expression was substantially downregulated in PCa cells LNCAP and C4-2B compared to PrEC (2A). Further, miR-149-5p expression analysis determine that miR-149-5p expression is consistently downregulated in metastatic CRPC tumors (n=30) (FIG. 2C) suggesting a potential tumor suppressor role of the miRNA.

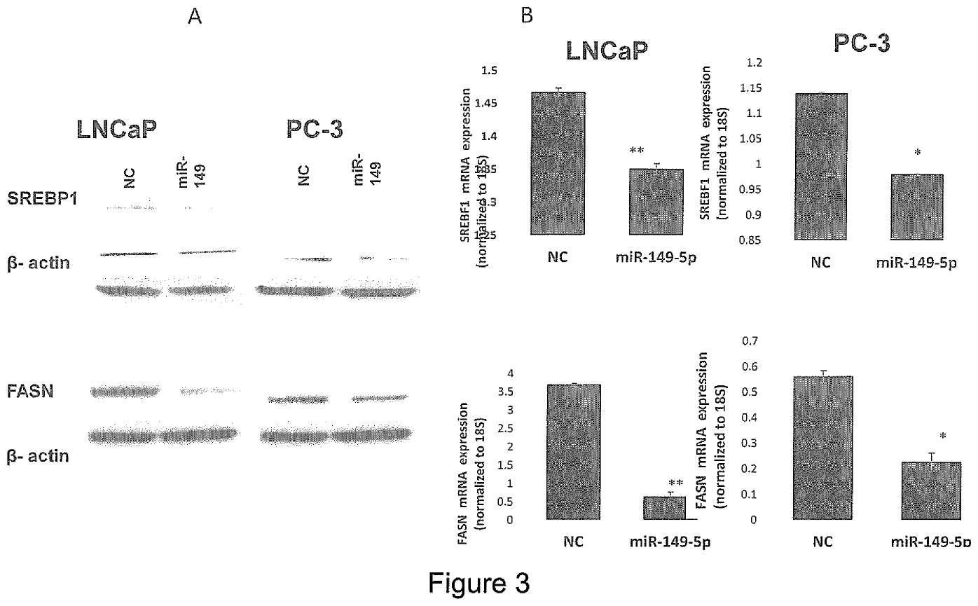

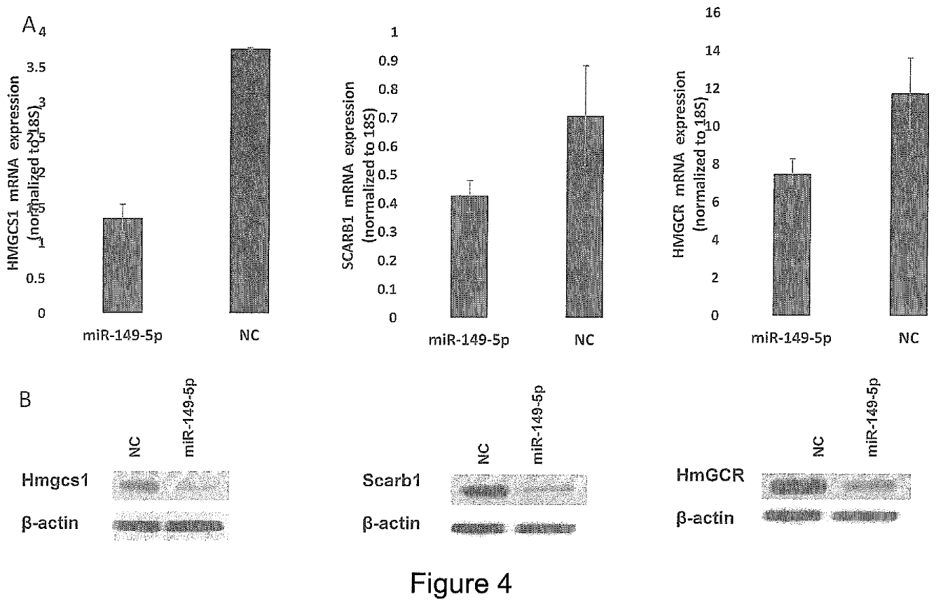

miR-149-5p ectopic expression results in the downregulated expression of endogenous SREBP1, HMGCS1, HMGCR and SCARB1 major proteins of lipid biosynthesis pathway: Since, pathway analysis of the miR-149-5p targets predicted above mentioned genes as targets, Applicants tested if miR-149-5p overexpression can experimentally downregulate SREBF1 expression and reduce the steady-state levels of SREBF1 mRNA. In miR-149-5p mimic transfected LNCaP, LAPC4 and 22RV1 cells Applicants found that SREBF1 expression was downregulated in all three cell lines (FIG. 3). Downregulation of SREBF1 function on miR-149-5p expression is also evident from the RT-qPCR and western blot data for HMGCS1, HMGCR and SCARB1 in LNCaP cell line (FIG. 4).

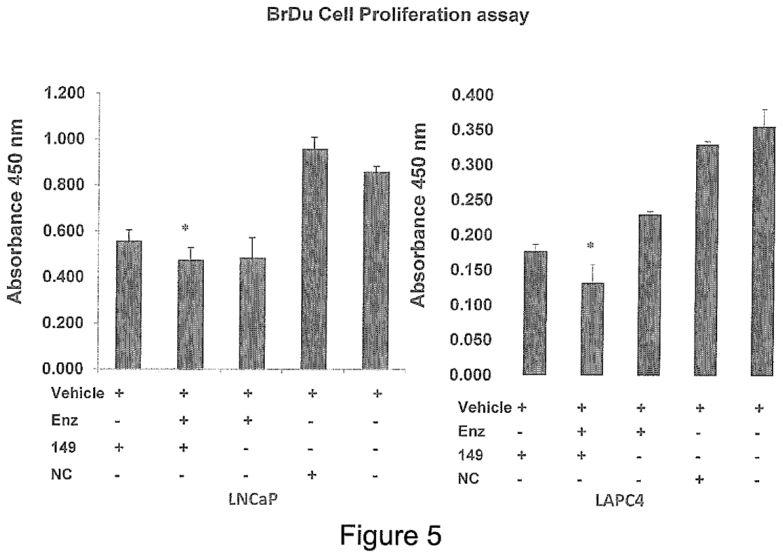

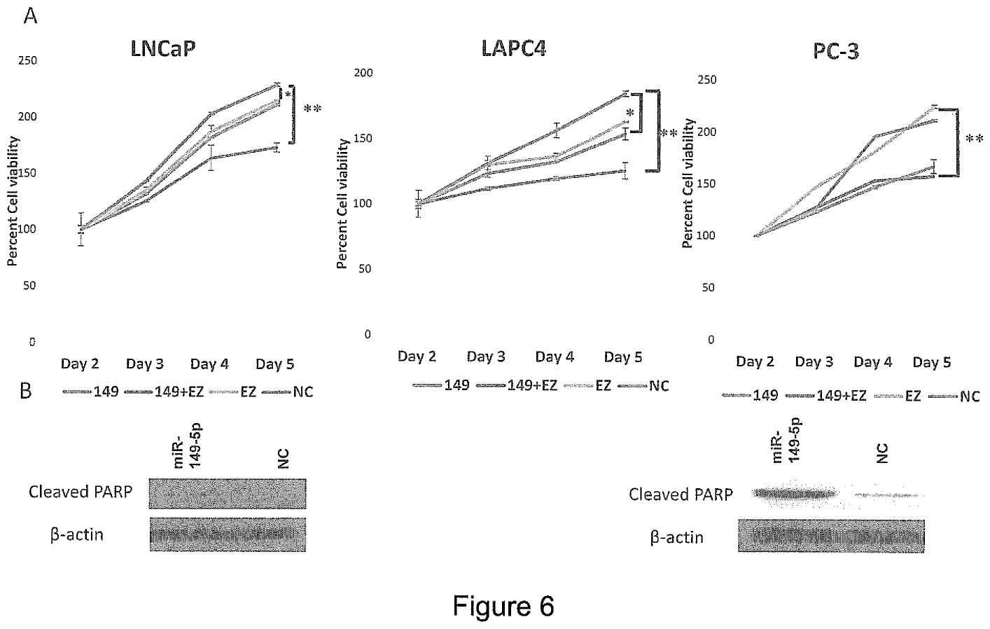

PCa cells sensitized by miR-149-5p, improves therapeutic action of enzalutamide: Applicants investigated if PCa cells can be sensitized by miR-149-5p and potentiate the Enz “therapeutic effect” in cell culture. Applicants performed a cell viability assay with and without miR-149-5p and Enz. LNCaP and LAPC4 cells were transfected with miR-149-5p mimic and treated with Enz. It is clearly evident (FIG. 6) using BrdU proliferation assay and (FIG. 5) cell viability assay that drug and miR combination significantly reduced cell viability. Applicants further checked if negative regulation of cell viability is through induction of apoptosis using cleaved PARP as marker for testing the apoptosis in miR-149-5p treated cells. Hence, this experiment lends credibility to our central hypothesis that miR-149-5p can potentiate the CRPC therapeutic effects of AR-antagonist Enz

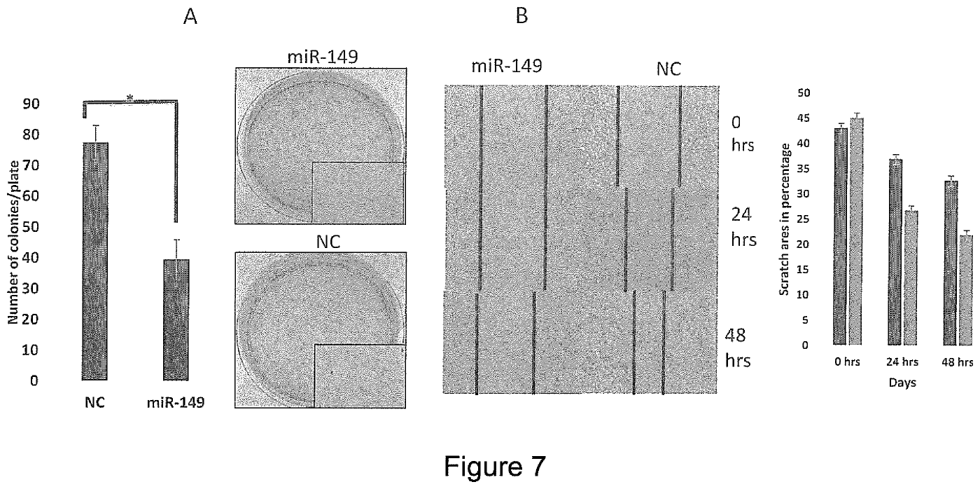

miR-149-5p inhibits PCa cell proliferation and migration: Next, Applicants tested if miR-149-5p is capable of inhibiting directional cell migration we performed a wound healing assay. LNCaP cells were transfected with the miRNA and “Wound” was created by removing the cell culture insert and the migration of cells were monitored at multiple time points by image analysis. As evident in the FIG. 7, miR-149-5p has substantially inhibited the migration and hampered the healing of wound in LNCaP cells (FIG. 7B left panel) as compared to negative control (FIG. 7B right panel). miR-149-5p also significantly reduced colony formation in 22RV1 cells treated with 20 nM miR-149-5p (FIG. 7A) suggesting the anti-proliferative effect of miR-149-5p.

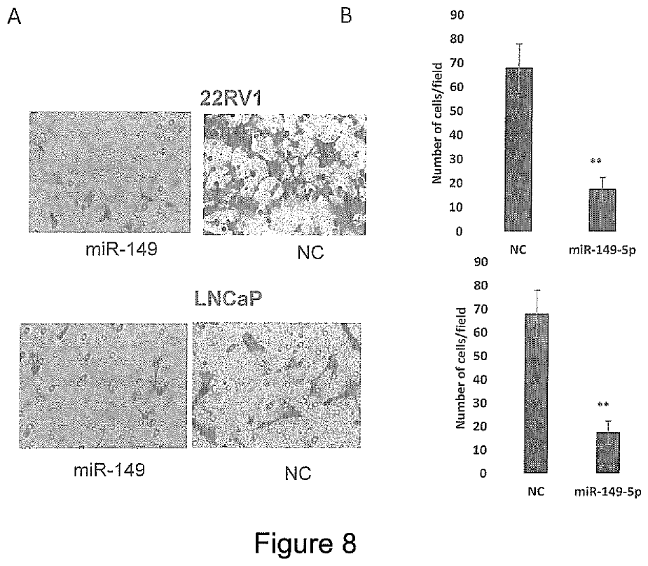

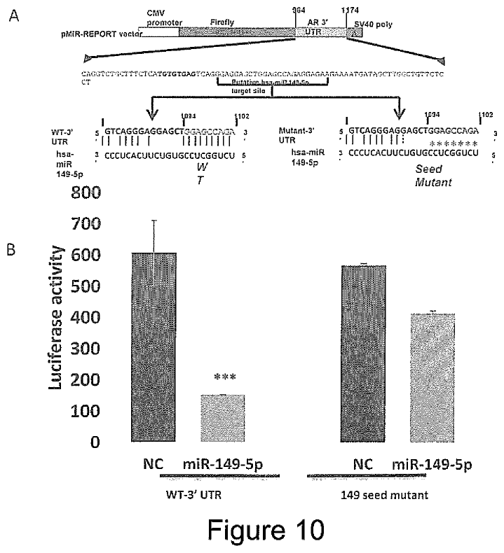

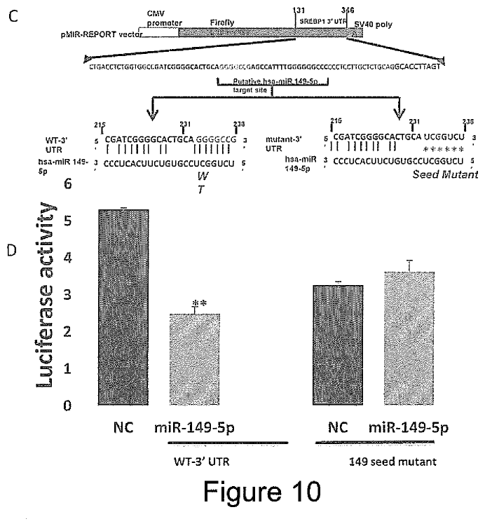

miR-149-5p inhibits PCa cell invasion: Next, Applicants investigated if miR-149-5p overexpression influences the invasive properties of LNCaP cells and 22RV1 cells. Applicants performed the Matrigel invasion assay of cells which were transfected with miR-149-5p mimic in DHT treated cells. FIG. 10 indicates that miR-149-5p was capable of impeding the invasive behavior of 22RV1 cells in both DHT untreated (FIG. 8 Panel B) and DHT treated cells (data not shown here) also show similar inhibition of invasion of LNCaP cells (data not shown). The cells that successfully invaded the Matrigel were quantified by image analysis (8 B). A high percentage of cells show inhibition of invasion as compared to the NC suggesting miR-149-5p successfully inhibited PCa cells invasion.

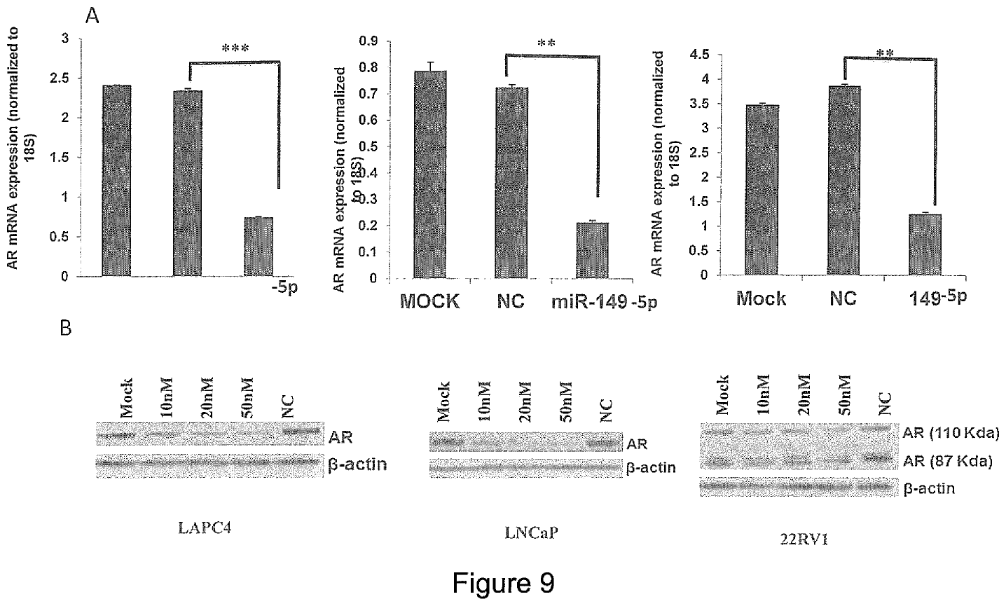

miR-149-5p downregulates expression of endogenous AR protein and mRNA in PCa cells: Applicants found that miR-149-5p mimic ectopic expression in LNCaP, LAPC4 and 22RV1 PCa cells targets AR expression and consequently, reduce the levels of AR protein and mRNA. Increasing concentration of miR mimics markedly reduced AR protein levels in three cell lines (FIG. 9A lanes as 10, 20 and 50 nM) but not in mock or negative control (NC) mimic cells (1^{st} and the last lanes, respectively in all three cell lines FIG. 9A). Interestingly, in 22RV1 cells both, 110 kDa full length WT and 87 kDa AR-V7 species appears to be targeted by the miRNA, a distinct advantage over Enz which cannot target the AR-V7 protein due to the lack of LBD. Thus, the general inventive concepts are based, in part, on the discovery that miR-149-5p can effectively suppress WT and truncated AR protein expression in both castration-sensitive and -refractory AR expressing PCa cells, by targeting the 3′UTR. Applicants also found the downregulation of AR mRNA in all three cell lines (FIG. 9B) confirming efficiency of miR-149-5p as a potential therapeutic molecule to target both isoforms of AR at posttranscriptional level.

miR-149-5p inhibits AR and SREBP1 expression by direct base-pairing to the 3′UTR: Target Validation: To test miR-149-5p and AR and SREBP1 3′UTR direct interaction we performed Firefly Luciferase (FFLuc) expression analysis by cloning miR149-5p predicted WT as well as seed mutant target sites in the AR 3′ UTR downstream of FFLuc open reading frame (ORF). In the mutant predicted seed miRNA:mRNA interaction was disrupted (FIG. 10, Panel A and C “WT and “Seed mut”). As shown in the FIG. 10 FFLuc expression was downregulated by miR-149-5p co-expression (FIG. 10B and D bar 1). However, miR-149-5p mediated FFLuc repression was rescued when AR or SREBP1 seed mutant was co-expressed with the miR mimic (10B and D bar 2). NC mimic has no effect on expression of FFluc with WT AR 3′ UTR, thus illustrating the fact that AR and SREBP1 are direct targets of miR-149-5p (FIG. 10B and D black bars)

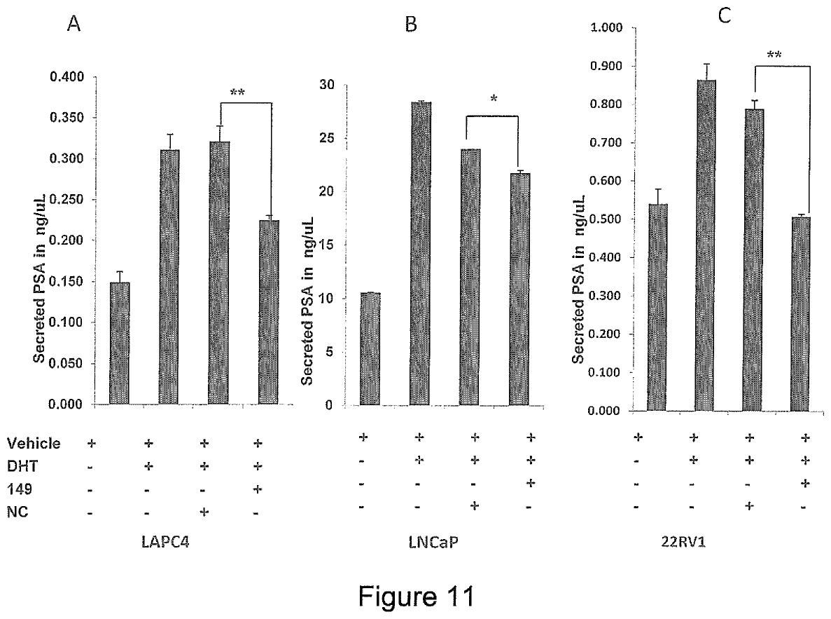

miR-149-5p overexpression represses the transactivation function of AR: Translocation of AR to nucleus trans-activates expression of numerous target genes containing androgen response elements (ARE). Human Kallikrein3 or Prostate specific antigen (PSA) is a direct target of AR and a biomarker of transactivation, therefore, we determined AR repression by miR-149-5p by measuring PSA expression in PCa cells both, at RNA and protein levels using qRT-PCR and PSA sandwich ELISA, respectively miR-149-5p overexpression downregulated the expression of PSA at both protein (FIG. 11), and mRNA levels (supplemental data) respectively in three cell lines) suggesting the therapeutic potential of miRNAs in targeting AR transactivation function.

miR-149-5p suppresses PCA xenograft growth by downregulating AR and proteins involved in cholesterogenesis. Given that miR-149-5p acted as a PCa tumor suppressor in vitro, Applicants further assessed its effect in vivo. As shown in FIG. 11 tumors treated with miR-149-5p grew at a much slower rate.

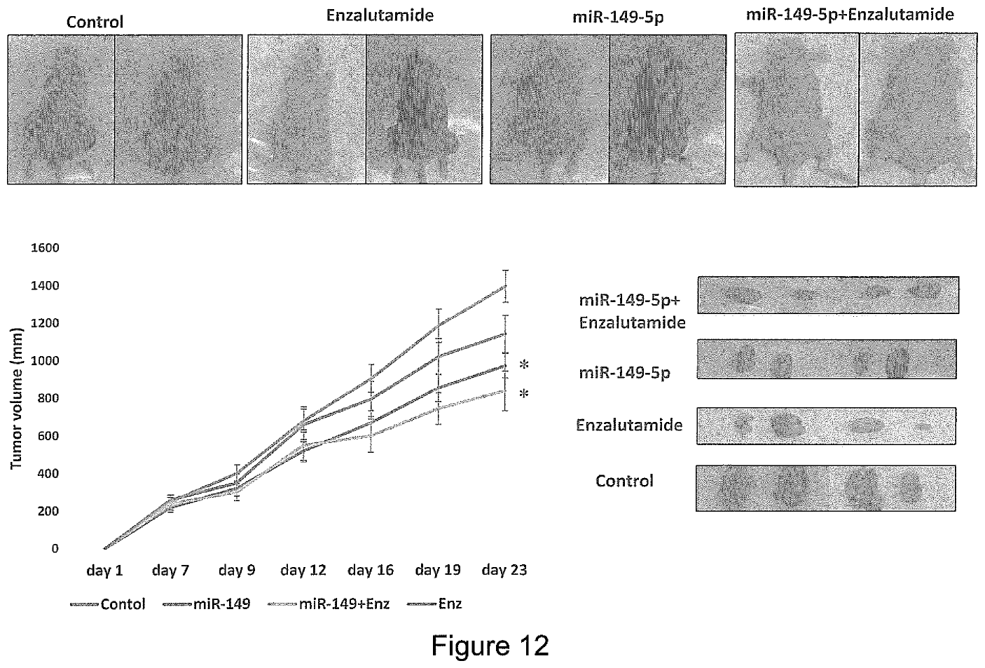

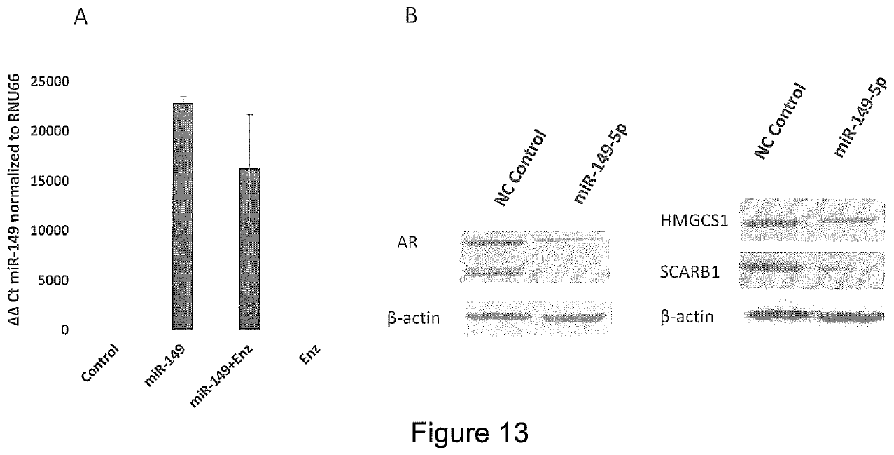

Significant reductions in tumor size were observed at the termination of experiment (P<0.05) this effect was further enhanced in tumors treated with combination of miR-149-5p and Enxalutamide. Moreover, in tumor tissues, miR-149-5p was significantly over-expressed in both the miR-149-5p treated as well as miR-149-5p and Enzalutamide treated tumors (FIG. 12) while the expression AR was obviously inhibited in miR-149-5p, we also noticed significant suppression of SCARB1 and HMGCS in the xenograft tumors (FIG. 13), indicating the recapitulation of the in-vitro results obtained in PCa cell line model.

miR-149-5p suppresses total intracellular cholesterol and testosterone on 22RV1 xenografts Tumors treated with miR-149-5p demonstrated significantly lower levels of intracellular cholesterol and testosterone compared the negative control treated tumors, as measured by ELISA (FIG. 14) and LC-MS data (supplementary materials) indicating that the reduction in expression of SCRAB1 and HMGCS rescues tumors of cholesterol accumulation and de novo synthesis that PCa tumors exhibit.

### Materials and Methods

In-silico analysis In order to investigate the significance of miR-149-5p downregulation in PCa progression we pooled out the predicted target genes of miR-149-5p from miRecord with selection criteria of a minimum of 4 prediction tools predicting the same targets. These genes were analyzed for their role in pathways critical for PCa growth. Pathways involved in PCa including AR and SREBF1 signaling pathways as well as steroid biosynthesis pathways were highly significant pathways predicted to be regulated by miR-149-5p using consensuspathwayDB.

Cell culture: Human PCa cell lines LNCaP, C4-2B and DU 145 were cultured in RPMI 1640 medium supplemented with 10% fetal bovine serum (FBS), 2 mM L-glutamine and antibiotics. CHO-K1 cells were cultured in Dulbecco's Modified Eagle's Medium (DMEM) supplemented with 5% FBS, 2 mM L-glutamine, 1 mM L-proline, 10 mM 4-(2-Hydroxyethyl)piperazine-1-ethanesulfonic acid (HEPES) and antibiotics. All cell lines were maintained in a humidified 5% CO2 atmosphere at 37° C. LNCaP, PC-3, 22RV1, and CHO-K1 cells were obtained from ATCC (Manassas, Va.) and LAPC4 were a generous gift from Dr. Nima Sharifi. C4-2B cells were obtained from ViroMed Laboratories (Minnetonka, Minn.).

Western blotting: LNCaP and PC-3 cells were seeded in six-well plates one day before transfection. Cells were transfected with synthetic hsa-miR-149 mimic or negative control (NC) miRNA mimic (Dharmacon, Chicago, Ill.) using Lipofectamine 2000 (Invitrogen, Carlsbad, Calif.) and harvested 48 hr post transfection for protein extraction. 10 micrograms of protein was resolved on NuPAGE 4-12% Bis-Tris gels and electro-transferred to nitrocellulose membranes. Following antibodies were used: mouse monoclonal anti-androgen receptor (AR) antibody (1:100, Santa Cruz Biotechnology, Santa Cruz, Calif.), SREBP1, FASN, HMGCR, HMGCS1 and SCARB1, mouse monoclonal anti-β-actin antibody (1:5000, Santa Cruz Biotechnology) and horseradish peroxidase conjugated anti-mouse secondary antibody (1:10,000, GE Healthcare, Piscataway, N.J.). Bands were detected using the ECL Plus Western blotting detection reagent (GE Healthcare). The signal intensities of bands were measured using the SCION IMAGE analysis software. The level of protein expression in each sample was determined by normalizing respective protein band intensity to β-actin band intensity.

Construction of reporter plasmids, transfections and luciferase assays: WT-3′ UTR (WT: wild type) reporter plasmid was constructed by cloning 120 base pairs (bp) fragment of AR and SREBP1 3′ UTR spanning the predicted target site for miR-149-5p downstream of firefly luciferase coding region in pMIR-REPORT vector (Ambion, Austin, Tex.). Site-directed mutagenesis of the putative target site for miR-149-5p in WT-3′ UTR construct was carried out to generate the MUT-3′ UTR construct. Nucleotide sequences of the constructs were confirmed by DNA sequencing. For luciferase assays, CHO-K1 cells (30,000 cells/well) were plated in 24-well plates one day before transfection. Cells were cotransfected using Lipofectamine 2000 (Invitrogen), with 100 ng of WT-3′ UTR or MUT-3′ UTR firefly luciferase reporter construct, 0.5 ng of renilla luciferase reporter plasmid (Promega, Madison, Wis.) and either miR-149-5p mimic (20 nM) or NC mimic (20 nM). Cell lysates were assayed for firefly and renilla luciferase activities 48 hr after transfection using the Dual-Luciferase Reporter Assay System (Promega) and Victor 3 Multilabel Counter 1420 (PerkinElmer). Renilla luciferase activity served as a control for transfection efficiency. Data are represented as ratio of firefly luciferase activity to renilla luciferase activity.

Quantitative real-time PCR analysis of miR-149-5p expression: Total RNA was isolated from PCa cells transfected with miR-149-5p mimic (20 nM) or NC mimic (20 nM). One microgram of DNased treated RNA was reverse transcribed into. First strand cDNA was synthesized from 10 ng of total RNA using primers specific for human mature miR-149-5p and rodent small nucleolar RNU66 (snoRNA66). Reverse transcription and quantitative real-time (qRT)-PCR was carried out using the TaqMan MicroRNA Reverse Transcription kit and TaqMan MicroRNA Assays (Applied Biosystems, Foster City, Calif.) as described previously (22). RNU-66 expression was used as an invariant control. The relative expression of miR-149-5p was calculated as ^{2-ΔCt} where ΔCt=Ct value of miR-149-5p in a sample—Ct value of snoRNA202 in that sample. Mean miR-149-5p expression±standard error (SE) was calculated from three independent experiments.

Determination of prostate specific antigen mRNA expression by qRT-PCR: Total RNA was isolated from LNCaP cells transfected with miR-149-5p mimic (20 nM) or NC mimic (20 nM) and also treated with DHT (20 nM) or vehicle control (dimethyl sulfoxide; DMSO). One microgram of DNase treated RNA was reverse transcribed into cDNA and real-time PCR reactions were set up as described previously. The specificity of amplification was confirmed by melting curve analysis and also by running PCR products on 3% agarose gels. Prostate specific antigen (PSA) mRNA expression was normalized to GAPDH mRNA expression. Mean normalized PSA expression±SE was calculated from three independent experiments.

Quantitation of PSA in cell culture supernatants: Amounts of secreted PSA protein in cell culture supernatants were determined by using the Quantikine human PSA kit (R&D Systems, Minneapolis, Minn.) according to the manufacturer's instructions. Serial dilutions of recombinant human PSA were used to plot the standard curve.

Cell viability assay: LNCaP and PC-3 cells were plated in six-well plates one day before transfection. The cells were transiently transfected with either miR-149-5p mimic (20 nM) or NC mimic (20 nM) using Lipofectamine 2000. Twenty-four hours after transfection, cells were seeded into 96-well plates at 5,000 cells/well. Cell viability was determined on 2nd, 4th and 5th day post transfection using CellTiter-Glo luminescent cell viability assay (Promega) according to the manufacturer's protocol.

Colony formation assays: LNCaP, and 22RV1 cells will be treated with 20nM miR-149-5p were split 24 hrs later and seeded in appropriate dilutions of agar for incubation at 37° C. for 3-4 weeks. Resultant colonies will be fixed by Gluteraldehyde, stained with crystal violet, photographed and counted/quantified by image analysis using Wimasis™ software.

Matrigel invasion assay: Migration and invasion assays were carried out using 8.0 μm Falcon Cell Culture with the Matrigel (BD Biosciences, San Jose, Calif., USA) as described previously.

Fluorimentry based total cholesterol assay: The Amplex Red Cholesterol assay was performed as per manufacturer's instructions, Amplex Red cholesterol assay Kit from thermo fisher (A12216). Tumor extracts were aliquoted in into a 96-well plate and Amplex Red reaction buffer was added. Aliquots of Amplex Red working solution with cholesterol esterase were added to duplicate wells and working solution without cholesterol esterase was added to the remaining duplicate wells to calculate the amount of free cholesterol. Plates were incubated for 30 min at 37° C. protected from light. Fluorescence was measured using an excitation wavelength of 560 nm and an emission wavelength of 590 nm. A cholesterol standard curve was also determined for each assay using a provided cholesterol reference standard. Cholesterol content was corrected by the amount of protein present in the lysate.

Estimation of tumor testosterone: the tumors were minced and homogenized the cells were pelleted down and supernatant was collected. Testosterone ELISA was performed using Enzo ELISA kit and results were normalized to the total protein level in each sample.

LC-MS assay. Tumors were quickly thawed and equal amounts of each sample was pulse homogenized using a water on ice. Homogenates were extracted similar to previously published methodology. 300 ug of protein were used for extraction of cholesterol and DHT, 450 ml MeOH, and 50 ml 1M NaOH added and vortexed followed by 1,500 ml MTBE and rotated for 30 min at room temperature. Water (400 ml) was added to affect phase separation and the top organic layer collected. Extraction was repeated by addition of 200 ml MeOH, 800 ml of MTBE, and 400 ml water. Extracts were first pooled, then split into two fractions 9:1 for later steroid:lipid analysis, and each aliquot dried with a centrifugal evaporator (Centrivap). The larger steroid fractions were redissolved in 0.5 ml acetonitrile, centrifuged, and the clear supernatant transferred to new tubes and dried again. The residue was dissolved in 50% MeOH, centrifuged, and the resulting clear supernatant extract derivatized by 4× dilution with 0.2 M Hydroxylamine and incubation for 1 hr at 65° C., similar to the method of Kalhorn et al. The smaller fraction was dissolved in 400 ml of AC:CHCl3 1:5, similar to the method of Liebisch et al.

Liquid Chromatography Tandem Mass Spectrometry (LC/MS/MS) Analysis of Cholesterol and d-DHT: Cholesterol and d-DHT were analyzed by the LC/MS/MS method using a Shimadzu LCMS-8050. standard solution or sample was injected onto a C18 column (Prodigy, 3 um, 2×150 mm, Phenomenex) for separation. Mobile phase A consisted of a water/formic acid (100/0.2 by volume); mobile phase B consisted of a methanol/formic acid (100/0.2 by volume). A linear gradient was generated at 0.2 ml/min: 0-2 min, 50% B; 2-8 min, 50%B to 100%B; 8-18 min, 100% B; 1-18.1 min, 100% B to 50%B, 18.1-26 min, re-equilibrate with 50% B. The injection volume was 5 ul. The column was controlled a 25 C and the autosampler compartment was set to 10 C. The HPLC eluent was injected on to the triple quadrupole mass spectrometer and ionized with electrospray ionization at positive mode. Both the cholesterol and d-DHT were monitored using selected reaction monitoring (SRM) mode. The SRM transitions (m/z) were 369-)147 for cholesterol and 306-)91 for d-DHT. The quantity of ADP and ATP was calculated using the external calibration curves.

As disclosed and suggested herein, the scope of the general inventive concepts are not intended to be limited to the particular exemplary embodiments shown and described herein. From the disclosure given, those skilled in the art will not only understand the general inventive concepts and their attendant advantages but will also find apparent various changes and modifications to the methods and systems disclosed. It is sought, therefore, to cover all such changes and modifications as fall within the spirit and scope of the general inventive concepts, as described and suggested herein, and any equivalents thereof.

What is claimed is:

1. A method of detecting, reducing, or treating therapeutic resistance associated with prostate cancer in an individual in need thereof, the method comprises administering a composition to increase a level of miR-149-5p in the individual.

2. The method of claim 1, wherein the individual exhibits one or more symptoms of castration-resistant prostate cancer.

3. The method of claim 2, wherein the individual has been diagnosed with castration-resistant prostate cancer.

4. The method of claim 1, wherein the individual has undergone androgen deprivation therapy.

5. The method of claim 1, wherein the composition comprises miR-149-5p.

6. The method of claim 1 further comprising administration of a pharmaceutical prostate cancer therapy.

7. The method of claim 1, wherein administration of the composition results in a reduction in tumor size and/or growth rate.

8. A composition for the treatment of prostate cancer or prostate cancer therapeutic resistance comprising a vector for increasing a level of miR149-5p in an individual.

9. The composition of claim 8, wherein the vector comprises miR149-5p.

10. A method of reducing intratumoral androgen synthesis in an individual experiencing one or more symptoms of prostate cancer, the method comprises administering a composition to increase a level of miR-149-5p in the individual.

11. The method of claim 10, wherein administration of the composition regulates at least one of cholesterogenesis and androgen receptor (AR) signaling.

12. The method of claim 10, wherein the individual exhibits one or more symptoms of castration-resistant prostate cancer.

13. The method of claim 12, wherein the individual has been diagnosed with castration-resistant prostate cancer.

14. The method of claim 10, wherein the individual has undergone androgen deprivation therapy.

15. The method of claim 10, wherein the composition comprises a vector for delivery of miR-149-5p.

16. The method of claim 10 further comprising administration of a pharmaceutical prostate cancer therapy.

17. The method of claim 10, wherein administration of the composition results in a reduction in tumor size and/or growth rate.
